(19) United States
(12) Patent Application Publication
(10) Pub. No.: US 2012/0070831 A1
(43) Pub. Date: Mar. 22, 2012

(54) Amplification of Distant Nucleic Acid Targets Using Engineered Primers

(75) Inventor: Jenny A. Johnson, Castro Valley, CA (US)

(73) Assignee: Roche Molecular Systems, Inc., Pleasanton, CA (US)

(21) Appl. No.: 13/237,645

(22) Filed: Sep. 20, 2011

Related U.S. Application Data
(60) Provisional application No. 61/385,210, filed on Sep. 22, 2010.

(51) Int. Cl. C12Q 1/68; C07H 21/04; C12N 9/12; C12P 19/34

(52) U.S. Cl. 435/6.11; 435/91.2; 536/24.33; 435/194

(57) ABSTRACT

The invention is a method of amplifying nucleic acids by synthesizing an engineered amplicon containing the sequence of interest, but omitting intervening sequences present in the template molecule. The method utilizes an “amplicon bridge” incorporated into the amplification primers. The length and content of the desired amplicon can be chosen by the operator and can contain unique regions for probe binding.

### CROSS-REFERENCE TO RELATED APPLICATIONS

This application claims the benefit of priority under 35 U.S.C. §119 to the Provisional Application Ser. No. 61/385,210, filed on Sep. 22, 2010.

### FIELD OF THE INVENTION

The invention relates generally to in vitro amplification and optionally, detection and quantification of nucleic acids. Specifically, the invention relates to a novel design of amplification primers that achieves bridging between two target nucleic acid sequences.

### BACKGROUND OF THE INVENTION

The polymerase chain reaction (PCR) has become a ubiquitous tool of biomedical research, disease monitoring and diagnostics. Amplification of nucleic acid sequences by PCR is described in U.S. Pat. Nos. 4,683,195, 4,683,202, and 4,965,188. PCR is now well known in the art and has been described extensively in the scientific literature. See PCR Applications, ((1999) Innis et al., eds., Academic Press, San Diego), PCR Strategies, ((1995) Innis et al., eds., Academic Press, San Diego); PCR Protocols, ((1990) Innis et al., eds., Academic Press, San Diego), and PCR Technology, ((1989) Erlich, ed., Stockton Press, New York). More recent improvements of the basic PCR technique involve new methods of detection, such as the “real-time” PCR assay as described in Holland et al., (1991) Proc. Natl. Acad. Sci. 88:7276-7280 and U.S. Pat. No. 5,210,015 and more recently, fluorescent probes, as described in U.S. Pat. No. 5,538,848.

One of the persistent challenges of the polymerase chain reaction has been amplification of longer stretches of the target nucleic acid. Unique enzymes capable of amplifying longer segments of DNA have been described (see U.S. Pat. Nos. 5,463,149 and 7,465,539). With mixtures of naturally occurring and engineered enzymes, it became possible to amplify up to 10 kb-long targets (see U.S. Pat. No. 5,512,462). Other attempts to cover long stretches of template involve the use of additional reagents in the reaction mixture and optimization of thermocycling profiles (see U.S. Pat. No. 5,512,462 and references cited therein). Nevertheless, even though amplification of longer sequences is possible, it remains technically challenging and costly. Yet the clinical users of PCR demand simple and low-cost protocols if an assay is to have a real-world diagnostic application. So far, hardly any long-PCR-based protocol has found its way into clinical practice.

Generally, amplification by PCR and design of amplification primers for any target sequence has become routine in the art. However, occasionally the nature of the target sequence is such that only a few suitable primer sites are available. These rare sites may be separated by long stretches of sequence. For example, in the case of pathogenic microorganisms, in order for PCR to generate an amplicon that is specific to only the targeted organism, primer sites must be chosen at highly conserved but target-specific regions of the genome. In microorganisms, sequence conservation sometimes occurs in “genomic islands” that are separated by large stretches of non-conserved sequences. For example, as described in IWG-CC, (2009) Classification of Staphylococcal Cassette Chromosome mec (SCCmec), guidelines for reporting novel SCCmec element, Antimicrob. Agents and Chem., 53:4961-4967, in S. aureus (MRSA), the distance between orfX and mecA loci varies between 2,000 and 20,000 by in different isolates. In Hepatitis C virus (HCV), the distance between the 5′- and 3′- Untranslated Regions (UTRs) is over 5,000 bp. In M. tuberculosis, the distance between suitable primer sites often exceeds 500 bp. With traditional primer design, these target sequences will yield amplicons too long for a conventional PCR profile and thus require a “long PCR” protocol. Although “long PCR” is routine in the investigative art, it is too costly and time consuming for clinical applications. The longer amplicons require special nucleic acid polymerases, adding cost to the assays. The longer amplicons also require longer extension times, making the assays run longer than traditional PCR assays. In clinical practice cheaper and faster assays are clearly preferable. The use of a generic PCR profile, often preprogrammed on the instruments by the vendor, is also desirable.

### SUMMARY OF THE INVENTION

The present invention provides for methods of detecting the presence or absence of a target nucleic acid sequence in a biological sample. In some embodiments, the method comprises:

contacting the target nucleic acid in the sample with a first pair of oligonucleotide primers flanking the first portion of the target sequence;

contacting the target nucleic acid in the sample with a second pair of oligonucleotide primers flanking the second portion of the target sequence; wherein one oligonucleotide primer in each pair is a bridge oligonucleotide containing a bridge sequence on the 5′-terminus; and

wherein the bridge sequence of the bridge oligonucleotide of the first pair is complementary to the bridge sequence of the bridge oligonucleotide of the second pair;

incubating the sample under the conditions permitting annealing of the oligonucleotide primers to the complementary sequences; and

incubating the sample under the conditions permitting extension of the oligonucleotide primers by the nucleic acid polymerase.

In other embodiments, the invention is a set of oligonucleotides for amplifying a target nucleic acid comprising: a first pair of oligonucleotides flanking the first portion of the target sequence; a second pair of oligonucleotides flanking the second portion of the target sequence; wherein one oligonucleotide in each pair contains a bridge sequence on the 5′-terminus; and wherein the bridge sequence of the oligonucleotide of the first pair is complementary to the bridge sequence of the oligonucleotide of the second pair.

In yet other embodiments, the invention is a kit for amplifying a target nucleic acid comprising: an amount of each of the oligonucleotides of the first pair, flanking the first portion of the target sequence; an amount of each of the oligonucleotides of the second pair, flanking the second portion of the target sequence; wherein one oligonucleotide in each pair contains a bridge sequence on the 5′-terminus; and wherein the bridge sequence of the oligonucleotide of the first pair is complementary to the bridge sequence of the oligonucleotide of the second pair; and a nucleic acid polymerase.

In other embodiments, the invention is a reaction mixture for amplifying a target nucleic acid comprising: a first pair of oligonucleotides flanking the first portion of the target sequence; a second pair of oligonucleotides flanking the second portion of the target sequence; wherein one oligonucleotide in each pair contains a bridge sequence on the 5′-terminus; and wherein the bridge sequence of the oligonucleotide of the first pair is complementary to the bridge sequence of the oligonucleotide of the second pair; a nucleic acid polymerase; and nucleoside triphosphates.

In other embodiments, the invention is a method of generating a control molecule for an amplification reaction, the method comprising: contacting the first target nucleic acid in the sample with a first pair of oligonucleotide primers flanking a portion of the first target sequence; contacting the second target nucleic acid in the sample with a second pair of oligonucleotide primers flanking a portion of the second target sequence; wherein one oligonucleotide primer in each pair is a bridge oligonucleotide containing a bridge sequence on the 5′-terminus; and wherein the bridge sequence of the bridge oligonucleotide of the first pair is complementary to the bridge sequence of the bridge oligonucleotide of the second pair; incubating the sample under the conditions permitting annealing of the oligonucleotide primers to the complementary sequences; and incubating the sample under the conditions permitting extension of the oligonucleotide primers by the nucleic acid polymerase.

### BRIEF DESCRIPTION OF THE DRAWINGS

FIG. 1 is a schematic representation of one embodiment of the invention as it applies to a double-stranded nucleic acid target.

FIG. 2 is a schematic representation of another embodiment of the invention as it applies to a double-stranded nucleic acid target.

FIG. 3 is a schematic representation of one embodiment of the invention as it applies to a single-stranded nucleic acid target.

FIG. 4 is a schematic representation of another embodiment of the invention as it applies to a single-stranded nucleic acid target.

FIG. 5 shows the results of applying the method of the present invention to detecting M. tuberculosis, where the products are detected by gel electrophoresis.

FIG. 6 shows the results of applying the method of the present invention to detecting M. tuberculosis, where the products are detected in real-time PCR.

FIG. 7 shows the results of applying the method of the present invention to detecting and identifying methicillin resistant and methicillin sensitive S. aureus and S. epidermidis.

### DETAILED DESCRIPTION OF THE INVENTION

### I. Definitions

The following definitions apply to the terms used throughout the application.

The term “amplicon” refers to a nucleic acid molecule that contains all or fragment of the target nucleic acid sequence and that is formed as the product of in vitro amplification by any suitable amplification method. For example, an amplicon can be formed via polymerase chain reaction (PCR) or ligase chain reaction (LCR).

The term “anchor sequence” within a primer sequence refers to a nucleic acid sequence that is substantially complementary to a portion of the target nucleic acid sequence so that primer annealing and primer extension may occur under suitable reaction conditions.

The term “bridge sequence” within a primer sequence refers to a nucleic acid sequence that is substantially complementary to the corresponding bridge sequence in another primer designed according to the method of the present invention, so that partial or complete annealing between the two bridge sequences on separate primers may occur under suitable reaction conditions.

The term “biological sample” refers to any composition containing or presumed to contain nucleic acid. The sample can be obtained by any means known to those of skill in the art. Such sample can be an amount of tissue or fluid, or a purified fraction thereof, isolated from an individual or individuals, including tissue or fluid, for example, skin, plasma, serum, whole blood and blood components, spinal fluid, saliva, peritoneal fluid, lymphatic fluid, aqueous or vitreous humor, synovial fluid, urine, tears, seminal fluid, vaginal fluids, pulmonary effusion, serosal fluid, organs, bronchio-alveolar lavage, tumors and paraffin embedded tissues. Samples also may include constituents and components of in vitro cell cultures, including, but not limited to, conditioned medium resulting from the growth of cells in the cell culture medium, recombinant cells and cell components. Samples may also include environmental samples and cultures of microorganisms, such as bacteria, protozoa and fungi and cultures containing viruses and mycoplasmae. Isolating nucleic acids from a biological sample is well known in the art.

The term “complementary nucleic acid” in relation to another nucleic acid means that at least a segment of one strand of the nucleic acid can combine or hybridize with at least a segment of one strand of another nucleic acid to form a duplex in which the nucleic acid strands are in an anti-parallel orientation. Complementary nucleic acid strands that anneal in an anti-parallel orientation are sometimes called “reverse complements” of each other. The duplex can be intramolecular, e.g., in the form of a hairpin loop of a single nucleic acid strand, or intermolecular, such as when two single-stranded nucleic acids hybridize with one another. A nucleic acid is “fully complementary” to a particular sequence when each base of the nucleic acid is complementary to the corresponding bases in the sequence. In some embodiments, complementarity is not perfect (i.e., nucleic acids can be “partially complementary” rather than “fully complementary”). Stable duplexes may contain mismatched base pairs or unmatched bases. “Substantially complementary” refers to a sequence that is at least 80% complementary to another sequence. It is understood that perfect complementarity is not required for the annealing between the two nucleic acid strands to occur. Furthermore, it is understood that perfect complementarity is not required for the extension of the annealed nucleic acid primer by a nucleic acid polymerase to occur. Therefore the term “complementary” includes nucleic acid strands that are perfectly complementary as well as substantially complementary, i.e. sufficiently complementary for the desired annealing or desired extension of the annealed nucleic acid primer by a nucleic acid polymerase to occur. “Complementary” therefore includes all degrees of complementarity between partially and completely complementary, e.g. 80%, 85%, 90%, 95%, 100% and the values in between.

The term “conserved nucleic acid region” refers to a region in the nucleic acid sequence that is highly similar or identical to nucleic acid sequences across species or among different molecules produced by the organisms of the same species.

The term “hybridization” is an interaction between two usually single-stranded or at least partially single-stranded nucleic acids. Hybridization occurs as a result of base-pairing between nucleobases and involves physicochemical processes such as hydrogen bonding, solvent exclusion, base stacking and the like. Hybridization can occur between fully-complementary or partially complementary nucleic acid strands. The ability of nucleic acids to hybridize is influenced by temperature, ionic strength and other hybridization conditions, which can be manipulated in order for the hybridization of even partially complementary nucleic acids to occur. Hybridization of nucleic acids is well known in the art and has been extensively described in Ausubel (Eds.) Current Protocols in Molecular Biology, v. I, II and III (1997).

The term “label” refers to a moiety attached (covalently or non-covalently), to a molecule, which moiety is capable of providing information about the molecule. Exemplary labels include fluorescent labels, radioactive labels, and mass-modifying groups.

The term “nucleic acid” refers to polymers of nucleotides (e.g., ribonucleotides and deoxyribonucleotides, both natural and non-natural) such polymers being DNA, RNA, and their subcategories, such as cDNA, mRNA, etc. A nucleic acid may be single-stranded or double-stranded and will generally contain 5′-3′ phosphodiester bonds, although in some cases, nucleotide analogs may have other linkages. Nucleic acids may include naturally occurring bases (adenosine, guanosine, cytosine, uracil and thymidine) as well as non-natural bases. The example of non-natural bases include those described in, e.g., Seela et al. (1999) Helv. Chim. Acta 82:1640. Certain bases used in nucleotide analogs act as melting temperature (Tₘ) modifiers. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303, which is incorporated herein by reference. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytidine; 5-fluorocytidine; 5-chlorocytidine; 5-iodocytidine; 5-bromocytidine; 5-methylcytidine; 5-propynylcytidine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like.

The term “long PCR” refers to polymerase chain reaction able to amplify targets at least 10 kb in length.

The terms “natural nucleotide” refer to purine- and pyrimidine-containing nucleotides naturally found in cellular DNA and RNA: cytosine (C), adenine (A), guanine (G), thymine (T) and uracil (U).

The term “non-natural nucleotide” or “modified nucleotide” refers to a nucleotide that contains a modified base, sugar or phosphate group, or that incorporates a non-natural moiety in its structure. The non-natural nucleotide can be produced by a chemical modification of the nucleotide either as part of the nucleic acid polymer or prior to the incorporation of the modified nucleotide into the nucleic acid polymer. In another approach a non-natural nucleotide can be produced by incorporating a modified nucleoside triphosphate into the polymer chain during enzymatic or chemical synthesis of the nucleic acid. Examples of non-natural nucleotides include dideoxynucleotides, biotinylated, aminated, deaminated, alkylated, benzylated and fluorophor-labeled nucleotides.

The term “nucleic acid polymerases” or simply “polymerases” refers to enzymes, for example, DNA polymerases, that catalyze the incorporation of nucleotides into a nucleic acid. Exemplary thermostable DNA polymerases include those from Thermus thermophilus, Thermus caldophilus, Thermus sp. ZO5 (see, e.g., U.S. Pat. No. 5,674,738) and mutants of the Thermus sp. ZO5 polymerase (see, e.g. U.S. patent application Ser. No. 11/873,896, filed on Oct. 17, 2007), Thermus aquaticus, Thermus flavus, Thermus filiformis, Thermus sp. sps17, Deinococcus radiodurans, Hot Spring family B/clone 7, Bacillus stearothermophilus, Bacillus caldotenax, Escherichia coli, Thermotoga maritima, Thermotoga neapolitana and Thermosipho africanus. The full nucleic acid and amino acid sequences for numerous thermostable DNA polymerases are available in the public databases.

An “oligonucleotide” refers to a short nucleic acid, typically ten or more nucleotides in length. Oligonucleotides are prepared by any suitable method known in the art, for example, direct chemical synthesis as described in Narang et al. (1979) Meth. Enzymol. 68:90-99; Brown et al. (1979) Meth. Enzymol. 68:109-151; Beaucage et al. (1981) Tetrahedron Lett. 22:1859-1862; Matteucci et al. (1981) J. Am. Chem. Soc. 103:3185-3191; or any other method known in the art.

The terms “polymerase chain reaction extension conditions” refer to conditions under which primers that hybridize to a template nucleic acid are extended by a polymerase during a polymerase chain reaction (PCR). In some instances, the extension conditions are identical to annealing conditions, i.e. the conditions under which the primers hybridize to the template nucleic acids. Those of skill in the art will appreciate that such conditions can vary, and are generally influenced by the nature of the primers and the template. Various PCR conditions are described in PCR Strategies (M. A. Innis, D. H. Gelfand, and J. J. Sninsky eds., 1995, Academic Press, San Diego, Calif.) at Chapter 14; PCR Protocols: A Guide to Methods and Applications (M. A. Innis, D. H. Gelfand, J. J. Sninsky, and T. J. White eds., Academic Press, NY, 1990).

A “primer” is an oligonucleotide which is capable of acting as a point of initiation of extension along a complementary strand of a template nucleic acid. A primer that is at least partially complementary to a subsequence of a template nucleic acid is typically sufficient to hybridize with template nucleic acid and for extension to occur.

Although other primer lengths are optionally utilized, primers typically comprise hybridizing regions that range from about 6 to about 100 nucleotides in length and most commonly between 15 and 35 nucleotides in length. The design of suitable primers for the amplification of a given target sequence is well known in the art and described in the literature cited herein. A primer nucleic acid can be labeled, if desired, by incorporating a label detectable by spectroscopic, radiological, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or otherwise known in the art. One of skill in the art will recognize that, in certain embodiments, primer nucleic acids can also be used as probe nucleic acids.

The term “primer extension” refers to the ability of a nucleotide incorporating biocatalyst, such as a polymerase, to add nucleotides to the 3′ terminus of a primer in a template-specific manner. A primer is non-extendible if, for example, the 3′ end of the primer is blocked.

As used herein, the term “probe” refers to an oligonucleotide (or other nucleic acid sequence) which can hybridize to a region of a target nucleic acid (or amplicon derived from such target nucleic acid), due to partial or complete complementarity of the probe and the target sequence. The probe can be labeled or unlabeled. The 3′-terminus of the probe optionally can be designed to prohibit extension of the probe by nucleic acid polymerase. This can be achieved, for example, by modifying the 2′-position or a 3′-position of the nucleotide at the 3′-terminus.

A “target nucleic acid sequence” or “target sequence” refers to a polynucleotide sequence to be amplified and optionally detected in a biological sample. The target nucleic acid can be, for example, the region of a nucleic acid that is fully or partially complementary to the primers as described herein. The target sequence can be of any length at least 10 nucleotides long. Target nucleic acids can be derived from essentially any source, including microorganisms, complex biological mixtures, tissues, bodily fluids, sera, preserved biological samples, environmental isolates, in vitro preparations or the like. The template or target may constitute all or a portion of a nucleic acid molecule.

A “thermostable nucleic acid polymerase” or “thermostable polymerase” is a polymerase enzyme which is relatively stable at elevated temperatures when compared, for example, to polymerases from E. coli. As used herein, a thermostable polymerase is suitable for use under temperature cycling conditions typical of the polymerase chain reaction (“PCR”).

A “melting temperature” or “Tₘ” refers to the temperature at which one half of a population of double-stranded (duplex) nucleic acid molecules, in homoduplexes or heteroduplexes becomes dissociated into single strands. The Tₘ of a duplex nucleic acid is affected by ionic strength and pH of the solution, as well as concentration, base composition and secondary structure of the nucleic acid itself. The Tₘ of a duplex under given conditions can be determined experimentally or predicted with the help of commercial software, such as Visual OMP™ (DNA Software, Inc., Ann Arbor, Mich.)

A “melting assay,” “melt assay,” or simply “a melt” is an assay in which the melting temperature (Tₘ) can be determined. In this assay, a duplex nucleic acid molecule is heated in a controlled temperature program, and the dissociation of the duplex into single strands is monitored by measuring a parameter, such as fluorescence, which changes with dissociation of the duplex. The melting data may be represented as a “melting curve,” i.e. a plot of fluorescence as a function of temperature (F vs. T). The melting data may also be represented as a “melting peak,” i.e. a plot of the rate of change in fluorescence over temperature interval as a function of temperature (dF/dT vs. T) or (−dF/dT vs. T), which typically has a parabolic shape. The Tₘ of the duplex is represented on a melting peak as the temperature value (T) at the apex of the parabola (dF/dT vs. T) or (−dF/dT vs. T).

### II. Introduction

The present invention is a method of synthesizing and amplifying nucleic acids using oligonucleotide primers. The method produces an engineered amplicon containing two or more target sequences, but omitting intervening sequences present in the template molecule. The engineered amplicon may also combine target sequences present on separate molecules. The desired result is accomplished by the use of an “amplicon bridge” incorporated into the amplification primers. Typically, the engineered amplicon of the present invention is short even though the upstream and downstream primer sites are far apart. The actual length of the desired amplicon can be chosen by the operator and can contain unique regions for probe binding.

The present invention enables amplification of target sequences where the primer sites are separated by large distances, using standard PCR reagents and PCR time and temperature profiles. The bridging allows amplification of targets with limited opportunities to design specific primers. The bridging also allows detection of several targets with a single probe in a single reaction. Furthermore, the bridging permits utilizing conserved regions of a target sequence that are separated by long variable regions. In some embodiments, the target sequences are located on separate nucleic acid molecules, e.g. separate chromosomes, separate microbial or viral genomes, separate extra-chromosomal elements or separate in vitro-generated nucleic acids.

This invention is not specific to any organism. The method may be applied to any PCR that requires a short amplicon for fast cycling times. In the art of detecting microorganisms, the method allows the user to target the most highly conserved primer sites for the best organism specificity and exclusivity without concern for amplicon length. In the case of eukaryotic targets, the method allows bypassing intervening sequences, such as introns or other sequences located between the target sites. For example, some mammalian genes, such as epidermal growth factor receptor (EGFR), tumor protein 53 (TP53), homeobox gene B7 (HoxB7), and many others, are often mutated at multiple sites in human tumors. (Kan et al., (2010) Diverse somatic mutation patterns and pathway alterations in human cancers, Nature doi:10.1038/nature09208.) The mutated sites are separated by large stretches of the non-mutant gene sequence. Therefore detecting multiple mutations typically involves using multiple sets of primers and probes to amplify and probe each mutation site. With the method of the present invention, the number of amplicons may be significantly reduced by “bridging” between separate sequences of interest.

In some embodiments, the method of the present invention is a method of generating a recombinant control molecule. Such a control molecule is an amplicon combining portions of two or more sequences of interest. In an assay where more than one potential target is to be detected (e.g. in the case where an infection with one or more of several pathogenic microorganisms or strains of microorganisms is suspected), such a recombinant control molecule can serve as a single positive control for all the targets to be detected.

The method is applicable to any nucleic acid target, e.g. DNA and RNA. Furthermore, the method enables different assays to use a generic detection probe.

### III. Primer and Amplicon Design

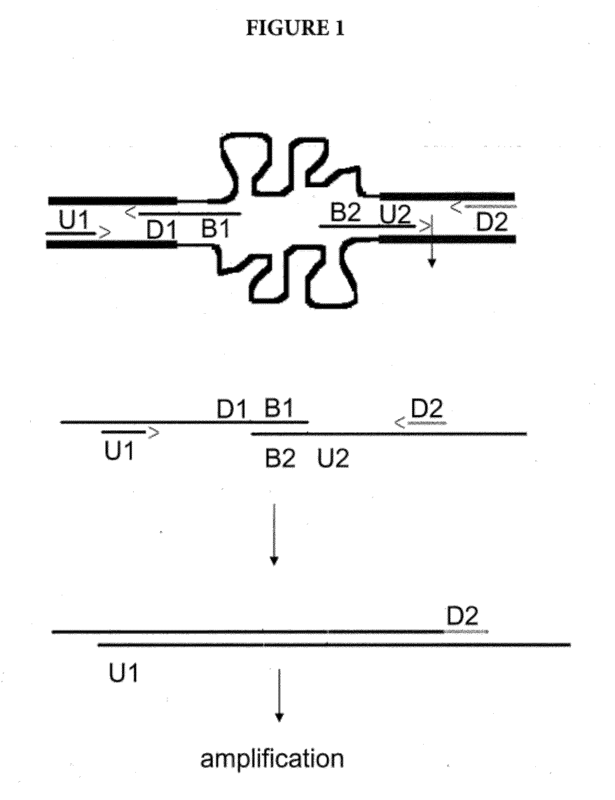

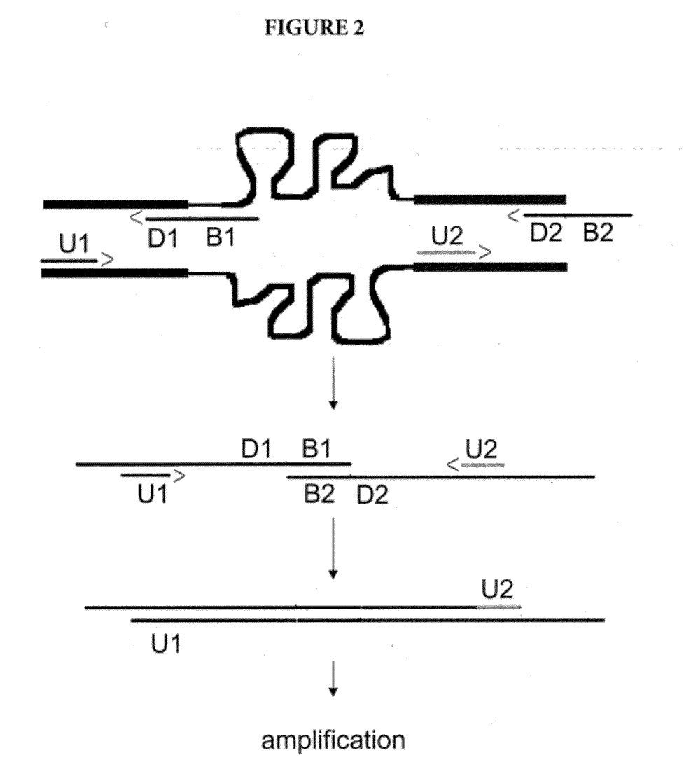

FIGS. 1 and 2 illustrate the embodiments of the method of the present invention as it applies to double-stranded nucleic acid target. The following abbreviations are used in FIGS. 1-4: U—upstream primer; D—downstream primer; B—bridge sequence at the terminus of an upstream or a downstream primer.

The method comprises the following steps described with reference to FIG. 1:

(1) designing at least two sets of upstream and downstream primers (U1 and D1, U2 and D2);

(2) designing a bridge sequence (B1) within one of the primers in the first set at the 5′-terminus of the primer (e.g., D1);

(3) designing a complement of the bridge sequence described in step 2 (B2) within one of the primers in the second set at the 5′-terminus of the primer (e.g., U2);

(4) incubating a sample containing at least two sets of upstream and a downstream primers and the template under the conditions suitable for annealing between the primers and their complementary sequences;

(5) incubating the sample from step (4) under conditions suitable for primer extension by a nucleic acid polymerase; and

(6) optionally, detecting the extension products generated in step (5).

In some embodiments, the conditions in steps (4) and (5) are identical.

As illustrated in FIG. 1, some primers of the present invention contain an anchor sequence at the 3′-end, which is substantially complementary to the template and a bridge sequence at the 5′-end, which is substantially complementary to another bridge sequence. In some embodiments of the invention, the primers in each set (e.g. U1 and D1, or U2 and D2) are separated by a short distance, typically 50-100 base pairs, although slightly shorter or longer distances are also possible. The distance between the first and the second primer set can be any distance. The first and a second primer set need not even be located on the same nucleic acid molecule or the same genome The bridge sequence (e.g., B1) may be between 20 and 40 base pairs-long, although slightly shorter or longer sequences are also possible. In one embodiment, the bridge sequence B1 is designed so that it is not complementary to any other sequence possibly present in the sample (other than B2), e.g., the patient sequence or the pathogen sequence.

The amplification primers of the present invention (aside from the bridge sequence) are oligonucleotides at least partially complementary to at least one of the existing variants of the target sequence. The length of the target binding sequence in each primer may range between 6 and 100 nucleotides, although most primers typically range between 15 and 35 nucleotides. The methods of optimizing the primers for nucleic acid amplification have been described for example, in PCR Protocols: A Guide to Methods and Applications, Innis et al., eds., (1990) Academic Press. Typically, primers are synthetic oligonucleotides, composed of A, C, G and T deoxyribonucleotides. However, unconventional base nucleotides and unconventional sugar nucleotides that can be incorporated into nucleic acids, can also be used in primers. For example, certain modified bases, when included in the target-binding portion of the primer are known to increase specificity of amplification, see U.S. Pat. No. 6,001,611. These modified bases include, without limitation, benzyl-, tert-butyl-benzyl or methyl modified deoxynucleotides, such as tert-butyl-benzyl-deoxyadenosine adenosine or benzyl-deoxycytidine, or methyl-adenosine as described in U.S. Pat. No. 6,001,611.

As illustrated in FIG. 1, the desired amplification product (a “bridge amplicon”) is an artificial sequence consisting of the portions U1-D1-B1/B2-U2-D2. The portion of the target sequence naturally present between sequences D1 and U2 is not included in the bridge amplicon.

As illustrated in FIG. 2, in some embodiments, the desired amplification product may comprise a different variation of the artificial sequence, for example, consisting of the sequence U1-D1-B1/B2-D2-U2. This sequence results from the bridge sequence and its complement being placed on the downstream primers of the first and the second set (D1 and D2). Other designs of the artificial sequence in the desired amplicon are also possible using variations of the method of the present invention. For example, the bridge sequence may be placed on an upstream primer of the first primer pair (U1), but on the downstream primer of the second primer pair (D2). In that case, the amplification product would consist of the sequence D1-U1-B1/B2 -D2-U2. Other such variations are possible as long as the bridge sequences (B1 and B2) can form a sufficiently stable duplex.

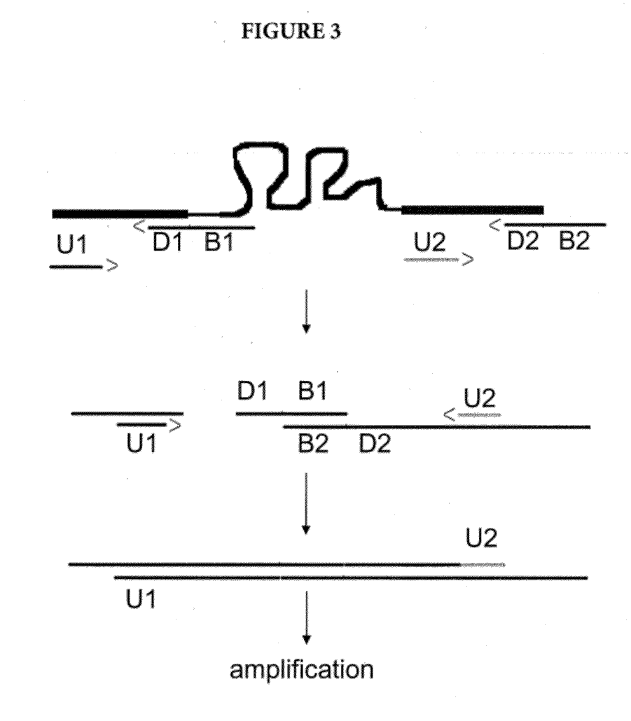

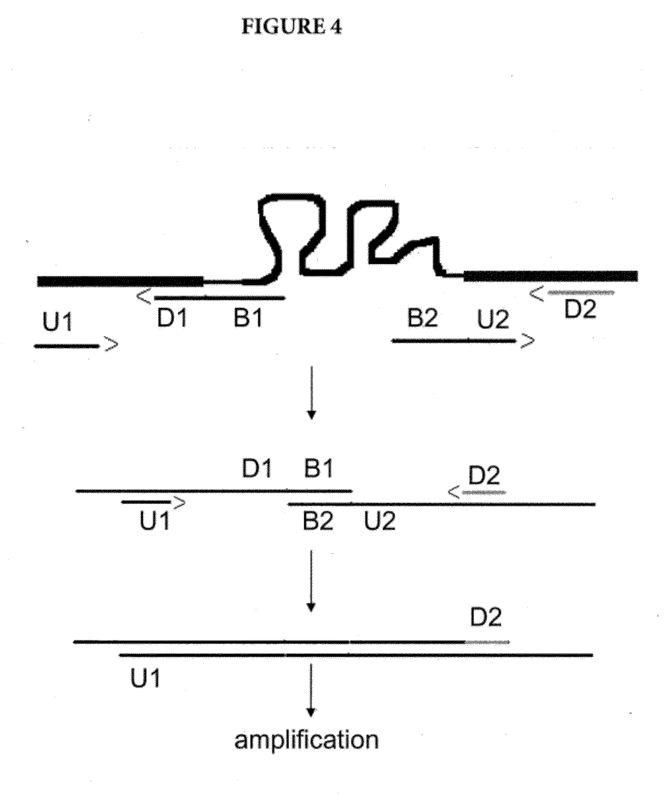

In other embodiments, the method of the present invention may be applied to amplification of single-stranded nucleic acids, such as RNA or single-stranded DNA, see FIGS. 3 and 4. In these embodiments, the primers used for reverse transcription or first strand synthesis may be conjugated to bridge sequences (FIG. 3). Alternatively, the primers used for second strand synthesis may be conjugated to bridge sequences (not shown). In yet another alternative, one of the primers used for reverse transcription or first strand synthesis and one of the primers used for second strand synthesis may be conjugated to bridge sequences (FIG. 4).

### IV. Assay Conditions

In some embodiments of the invention, the PCR amplification is conducted under the conditions that disfavor generation of amplicons other than the bridge amplicon. For example, in some embodiments of the invention, generation of the very short extension products (e.g., U1-D1 and U2-D2, see FIG. 1) may be discouraged by limiting the concentration of bridge primers (D1 and U2, FIG. 1) relative to the concentration of the “outer” primers (U1 and D2, FIG. 1) in the reaction mixture. In other embodiments of the invention, generation of very long extension products containing the entire intervening sequence (e.g., between U1 and D2, FIG. 1) may be discouraged by using shorter extension times. An exemplary thermocycling protocol would commence with one or more cycles with unusually long extension time, to be followed by multiple cycles with shorter extension times. These modifications of the thermocycling profile are optional and may not be necessary in all instances of practicing the invention.

Any thermostable nucleic acid polymerase can be used in the method of the present invention. Various thermostable nucleic acid polymerases are known in the art. Sometimes it is advantageous to use a polymerase lacking the 5′-3′ nuclease activity. It is sometimes desirable to use a polymerase without the proof-reading (3′-5′-exonuclease) activity. It may also sometimes be desirable to have an enzyme with a “hot start” capability, such as the reversibly modified enzymes described in U.S. Pat. Nos. 5,677,152 and 5,773,528.

### V. Detection

The amplicons generated by the method of the present invention may be detected by any method known in the art. The means of detection may be specific to the target sequence, or may be non-specific, i.e. generic to all double stranded DNA.

For example, the amplification products may be detected after the amplification has been completed, for example, by gel electrophoresis of the unlabeled products and staining of the gel with a nucleic acid-binding dye. Alternatively, the amplification products may carry a fluorescent, radioactive or a chemical label, either by virtue of incorporation during synthesis or by virtue of being the extension products of a labeled primer. After, or during electrophoresis, the labeled amplification products may be detected with suitable radiological or chemical tools known in the art. After electrophoresis, the product may also be detected with a target-specific probe labeled by any one of the methods known in the art. The labeled probe may also be applied to the target without electrophoresis, i.e. in a “dot blot” assay or the like.

In other embodiments, the presence of the amplification product may be detected in a homogeneous assay (also known as real-time PCR), i.e. an assay where the nascent product is detected during the cycles of amplification, or at least in the same unopened tube, and no post-amplification handling is required. A typical real-time PCR protocol involves the use of a labeled probe, specific for each target sequence. The probe is preferably labeled with one or more fluorescent moieties, which emit light of a detectable wavelength. Upon hybridizing to the target sequence or its amplicon, the probe exhibits a detectable change in fluorescent emission. A homogeneous amplification assay has been described for example, in U.S. Pat. No. 5,210,015. Homogeneous amplification assays using nucleic acid-intercalating dyes have been described for example, in U.S. Pat. Nos. 5,871,908 and 6,569,627. The homogeneous assay may also employ fluorescent probes labeled with two interacting fluorophores, such as “molecular beacon” probes (Tyagi et al., (1996) Nat. Biotechnol., 14:303-308) or fluorescently labeled nuclease probes (Livak et al., (1995) PCR Meth. Appl., 4:357-362).

In some embodiments, the amplification product may be detected using a specific probe that hybridizes to the amplification product (FIG. 1). The design of probes for real-time PCR or post-PCR detection, including labeled probes, is well known in the art and has been described for example, in Livak et al., (1995) Genome Res. 4:357-362.

The probe oligonucleotides can be labeled by incorporating one or more chromophores. A single chromophore, which is a fluorophore, may be used as described in a U.S. application Ser. No. 12/330,694 filed on Dec. 9, 2008. Where two chromophores are used both chromophores may be fluorophores or one of the chromophores may be a non-fluorescent quencher. Examples of suitable fluorophores include dyes of the fluorescein family (FAM, HEX, TET, JOE, NAN and ZOE), rhodamine family (Texas Red, ROX, R110, R6G and TAMRA), cyanine family (Cy2, Cy3, Cy5 and Cy7) coumarin family, oxazine family, thiazine family, squaranine family and other families of fluorescent dyes suitable for the labeling and detection of nucleic acids. The second chromophore may be incorporated into the same probe oligonucleotide or a separate probe oligonucleotide. Commonly used dark quenchers include Black Hole Quenchers™ (BHQ), (Biosearch Technologies, Inc., Novato, Calif.), Iowa Black™, (Integrated DNA Tech., Inc., Coralville, Iowa), and BlackBerry™ Quencher 650 (BBQ-650), (Berry & Assoc., Dexter, Mich.).

In some embodiments, a single probe is labeled with two chromophores forming a FRET pair. In some embodiments, both chromophores are fluorophores. In other embodiments one chromophore is a non-fluorescent quencher. The chromophores forming the FRET pair may be conjugated to the same or separate probe molecules. The use of FRET probes in a melting assay has been described in U.S. Pat. No. 6,174,670 and in De Silva et al., (1998) “Rapid genotyping and quantification on the LightCycler™ with hybridization probes,” Biochemica, 2:12-15. In other embodiments, the probe is labeled with a single chromophore that interacts with a second chromophore either conjugated with or intercalated into the target nucleic acid. See U.S. Pat. No. 5,871,908.

In some embodiments, an amplification product may also be identified by virtue of its distinctive melting temperature, see U.S. Pat. Nos. 5,871,908 and 6,569,627. A melting assay involves determining the melting temperature (melting point) of a double-stranded target, or a duplex between the labeled probe and the target. As described in U.S. Pat. No. 5,871,908, to determine melting temperature using a fluorescently labeled probe, a duplex between the target nucleic acid and the probe is gradually heated (or cooled) in a controlled temperature program. The dissociation of the duplex results in a change in proximity of the interacting fluorophores or fluorophore and quencher. The interacting fluorophores may be conjugated to separate probe molecules, as described in U.S. Pat. No. 6,174,670. Alternatively, one fluorophore may be conjugated to a probe, while the other fluorophore may be intercalated into a nucleic acid duplex, as described in U.S. Pat. No. 5,871,908. As yet another alternative, the fluorophores may be conjugated to a single probe oligonucleotide. Upon melting of the duplex, the fluorescence is quenched as a result of the change in secondary structure of the now single-stranded probe that brings together the fluorophore and the quencher.

The melting of the nucleic acid duplex is monitored by measuring the associated change in fluorescence. The change in fluorescence may be represented on a graph referred to as a “melting profile.” Because different probe-target duplexes may be designed to melt (or reanneal) at different temperatures, each probe will generate a unique melting profile. Properly designed probes would have melting temperatures that are clearly distinguishable from those of the other probes in the same assay. Many existing software tools enable one to design probes for a same-tube multiplex assay with these goals in mind. For example, Visual OMP™ software (DNA Software, Inc., Ann Arbor, Mich.) enables one to determine melting temperatures of nucleic acid duplexes under various reaction conditions.

The design of hybridization probes for detecting nucleic acids is known in the art. The same probe may serve as a hybridization probe or a melt probe or both. Whether the probe is to serve as a melt probe, a single hybridization probe or a member of a pair of hybridization probes, the design of the probe oligonucleotide is guided by the same principles, known in the art and described herein. See, e.g., Saiki, et al., 1986. Nature 324: 163; Saiki, et al., 1989. Proc. Natl. Acad. Sci. USA 86: 6230. These design principles may be applied manually or with a help of software.

### VI. Sets and Kits

In another embodiment, the invention is a set of oligonucleotides for amplifying a target nucleic acid comprising: a first pair of oligonucleotides flanking the first portion of the target sequence; a second pair of oligonucleotides flanking the second portion of the target sequence; wherein one oligonucleotide in each pair contains a bridge sequence on the 5′-terminus; and wherein the bridge sequence of the oligonucleotide of the first pair is complementary to the bridge sequence of the oligonucleotide of the second pair. In some embodiments, the set of oligonucleotides of the present invention further comprises one or more probe oligonucleotides.

In another embodiment, the invention is a kit for amplifying a target nucleic acid comprising: an amount of each of the oligonucleotides of the first pair, flanking the first portion of the target sequence; an amount of each of the oligonucleotides of the second pair, flanking the second portion of the target sequence; wherein one oligonucleotide in each pair contains a bridge sequence on the 5′-terminus; and wherein the bridge sequence of the oligonucleotide of the first pair is complementary to the bridge sequence of the oligonucleotide of the second pair; an amount of nucleic acid polymerase, an amount of each of the four nucleic acid precursors, i.e. nucleoside triphosphates, and an amount of organic and inorganic ions, suitable for the support of the activity of the nucleic acid polymerase. In some embodiments, the kit further comprises as a control, an amount of the target sequence, for example an amount of DNA isolated from the pathogen to be detected. In some embodiments, the kit further comprises as a control, an amount of the synthetic amplicon incorporating the bridge sequence. Optionally, the kit may also contain a pyrophosphatase for minimizing pyrophosphorolysis of nucleic acids; a uracil N-glycosylase (UNG) for protection against carry-over contamination of amplification reactions; and a set of instructions for conducting amplification according to the present invention.

### VII. Reaction Mixtures

In another embodiment, the invention is a reaction mixture for amplifying a target nucleic acid comprising: a first pair of oligonucleotides flanking the first portion of the target sequence; a second pair of oligonucleotides flanking the second portion of the target sequence; wherein one oligonucleotide in each pair contains a bridge sequence on the 5′-terminus; and wherein the bridge sequence of the oligonucleotide of the first pair is complementary to the bridge sequence of the oligonucleotide of the second pair; a nucleic acid polymerase, nucleic acid precursors, i.e. nucleoside triphosphates, and organic and inorganic ions, suitable for the support of the activity of the nucleic acid polymerase. In some embodiments, the reaction mixture further comprises probes for the detection of the amplified nucleic acids.

### VIII. Examples

As an illustration only and not to limit the scope of the invention, the method was applied to microorganisms.

### Example 1

### Detection of a Pathogen Sequence (M. Tuberculosis) by Generating and Detecting an Amplicon Bridging Distant Sequences

In this example, the method was used detect the presence of M. tuberculosis (MTB) DNA in the sample. The example illustrates an MTB amplicon from partially overlapping oligos that bypasses the 564 by distance between highly conserved gene regions and generates a short amplicon (233 bp). The amplicon is tailored to the exact length desired and is designed to contain a TaqMan® probe-binding region.

For this example, the upstream and downstream primers and probes were selected from Table 1.

Each 50 μL amplification reaction contained 15 mM Tris, pH 8.5, 77 mM Tricine, 55 mM potassium hydroxide, 95 mM potassium acetate, 9.5% Glycerol (v/v), 1.15% DMSO, 0.58 mM each dATP, dCTP, dGTP and dUTP, 0.5 μM each upstream and downstream assay oligonucleotides, 0.1 μM upstream and downstream bridge oligonucleotides, 0.093 μM probe, 154 U/mL ZO5 DNA polymerase, 75 U/mL UNG, 0.045% sodium azide (w/v), pH 8.50. The reactions were subjected to the following temperature profile: 2 min at 50° C., followed by five cycles of 95° C. for 5 sec and 55° C. for 3 min, followed by 55 cycles of 91° C. for 5 sec and 58° C. for 15 sec. The amplification and detection were performed using the LightCycler®480 instrument (Roche Applied Science, Indianapolis, Ind.).

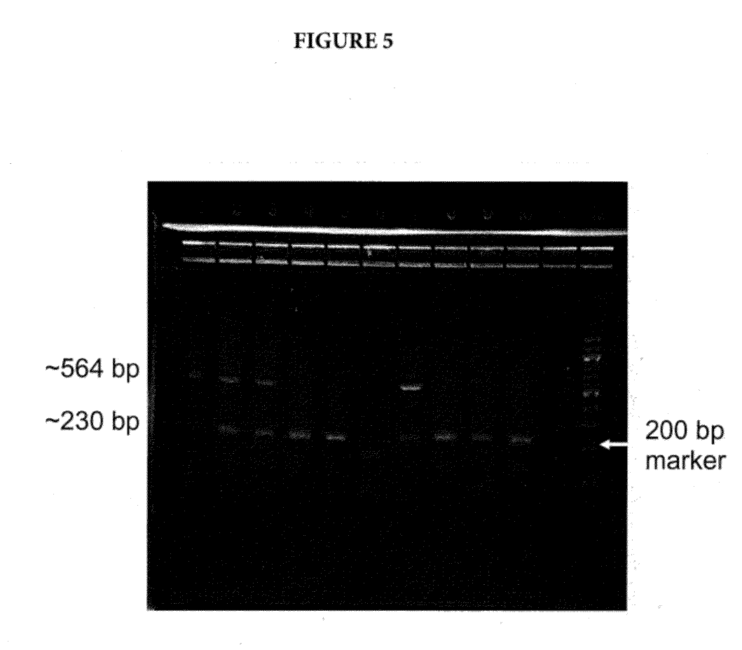

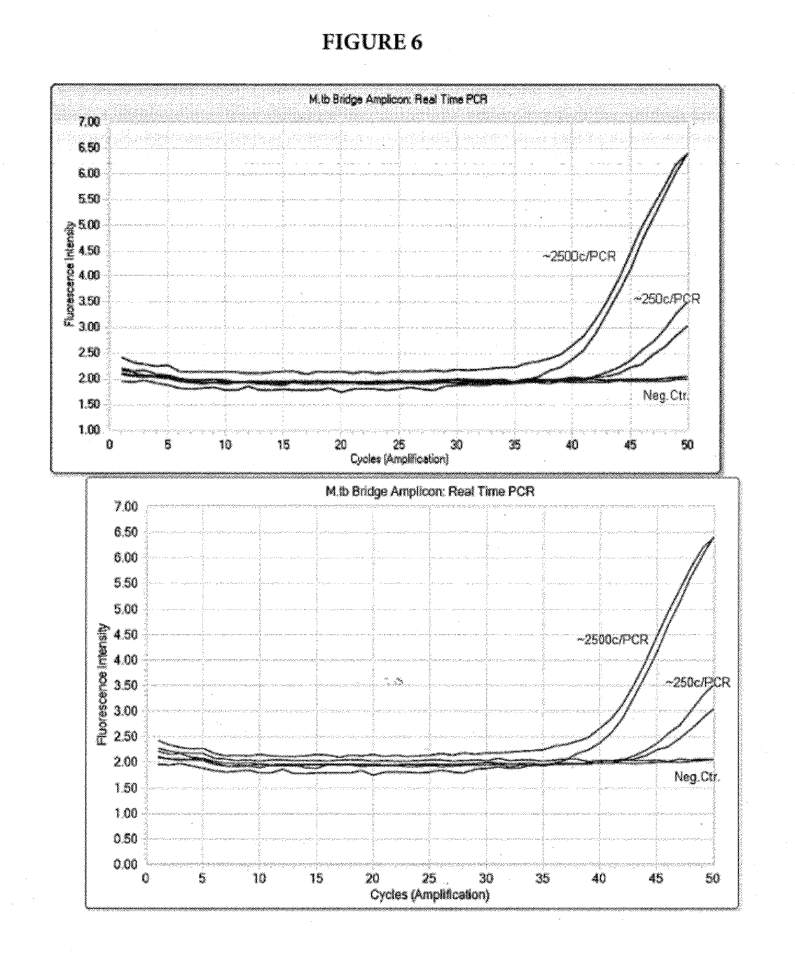

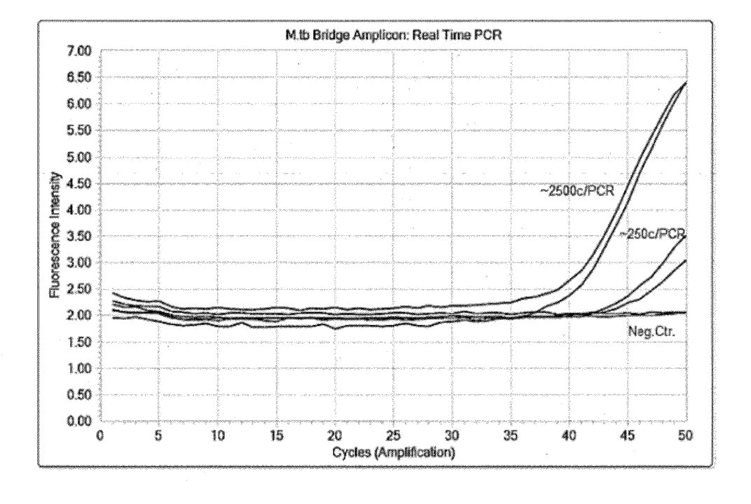

The amplification products were analyzed by gel electrophoresis. Results are shown on FIGS. 5 and 6. The results show that an amplicon having the predicted size of the bridge amplicon (230 bp) is indeed observed. The results also show that under suboptimal reaction conditions, the read-through amplicon (564 bp) is also observed. The desired amplicon is also detectable in a real-time PCR assay as shown on FIG. 6. The growth curves were observed for the reaction with the original input of 250 copies of the template sequence and with the original input of 2,500 copies of the template sequence. The nucleotide sequence of the bridge amplicon was determined and found to correspond to the predicted sequence of the bridge amplicon.

TABLE 1
Primers and probes for example 1

| Sequence | Function | Sequence 5′-3′ |
|---|---|---|
| SEQ ID | upstream | TAACACATGCAAGTCGAACGGAAA |
| NO: 1 | primer | |
| SEQ ID | upstream | CGTGCTTAACACATGCAAGTC |
| NO: 2 | primer | |
| SEQ ID | upstream | TGCTTAACACATGCAAGTCGAAC |
| NO: 3 | primer | |
| SEQ ID | upstream | ATGCAAGTCGAACGGAAAGGTC |
| NO: 4 | primer | |
| SEQ ID | downstream | AAGCGCTATGGCATGCGCCATAGCATAATCGGATTGACGGTAGGTGG |
| NO: 5 | bridge | AGAAG |
| | primer | |
| SEQ ID | downstream | AAGCGCTATGGCATGCGCCATAGCATAATGGTGCACATTGCCCAGGA |
| NO: 6 | bridge | AATTTTCGGATTGACGGTAGGTGGAGAAG |
| | primer | |
| SEQ ID | upstream | TTATGCTATGGCGCATGCCATAGCGCTTTTCGCCACTCGAGTACCTCC |
| NO: 7 | bridge | GAAG |
| | primer | |
| SEQ ID | upstream | TTATGCTATGGCGCATGCCATAGCGCTTTGGTGCACATTGCCCAGGA |
| NO: 8 | bridge | AATTTTTCGCCACTCGAGTACCTCCGAAG |
| | primer | |
| SEQ ID | downstream | CAGTTAAGCTGTGGGATTTCACGAACA |
| NO: 9 | primer | |
| SEQ ID | downstream | TTAAGCCGTGAGATTTCACGAACAAC |
| NO: 10 | primer | |
| SEQ ID | downstream | TCACAGTTAAGCCGTGAGATTTCAC |
| NO: 11 | primer | |
| SEQ ID | downstream | ACGCTCACAGTTAAGCCGTGAGA |
| NO: 12 | primer | |
| SEQ ID | Probe | RCAACTAQCGTGCCAGCAGCCGCP |
| NO: 13 | | |
| SEQ ID | Probe | RCGTGCCQAGCAGCCGCGGTAATP |
| NO: 14 | | |
| SEQ ID | Probe | RAGCAGCQCGCGGTAATACGTAGGP |
| NO: 15 | | |

R—reporter fluorophore
Q—quencher fluorophore
P—3′-phosphate group

In this example, the method of the present invention achieved bridging amplification between target sequences separated by 564 base pairs and shortened the amplicon to 230 base pairs.

### Example 2

### Detection of the Pathogen Sequence (MRSA) by Generating and Detecting an Amplicon Combining Targets that are Further Apart on the Genome

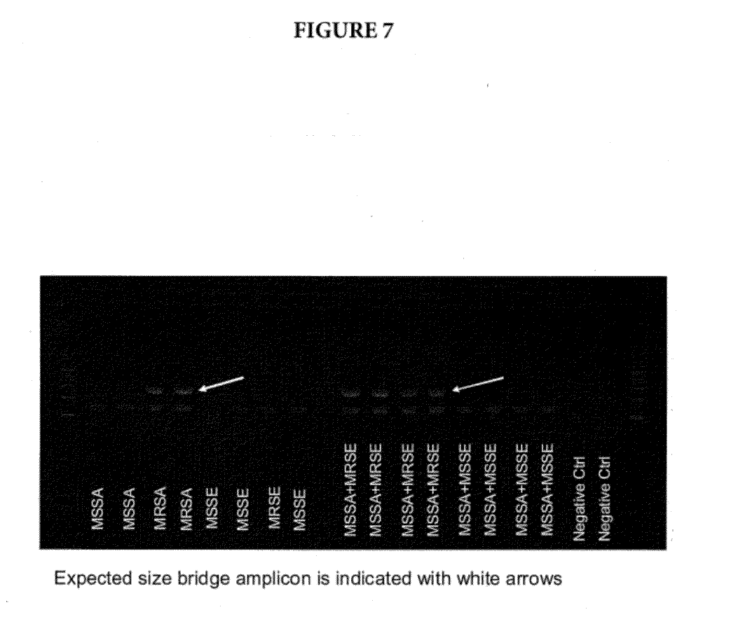

In another example, the method of the present invention was successfully applied to S. aureus (MRSA). In that example, the target sequences orfX and mecA are separated by distances ranging from 2,500 to 50,000 base pairs, depending on the strain. Therefore, without the method of the present invention, the amplicon size would be not only unmanageably large, but also variable. The method of the present invention was able to bridge that distance and generate a short amplicon in all MRSA strains tested. Results are shown in FIG. 7.

For this example, the upstream and downstream primers were selected from Table 2. The reaction conditions were identical to those in the first example, and the cycling conditions were as follows: 2 min at 50° C., followed by 94° C. for five sec, followed by 55° C. for 2 min, followed by 60° C. for 6 min, followed by 65° C. for 4 min, followed by 5 cycles of 95° C. for 5 sec and 55° C. for 30 sec, followed by 45 cycles of 91° C. for 5 sec and 58° C. for 25 sec.

TABLE 2
Primers for example 2

| Sequence | Function | Sequence 5′-3′ |
|---|---|---|
| SEQ ID | upstream | GATCAAACGGCCTGCACAA |
| NO: 16 | primer | |
| SEQ ID | upstream | CCTGCACAAGGACGTCTTACAA |
| NO: 17 | primer | |
| SEQ ID | upstream | GGCCTGCACAAGGACGTC |
| NO: 18 | primer | |
| SEQ ID | upstream | CACAAGGACGTCTTACAACGC |
| NO: 19 | primer | |
| SEQ ID | downstream | AAGCGCTATGGCATGCGCCATAGCATAATGACTGAACGTCCGATAAAAATAT |
| NO: 20 | bridge | ATAATAGTTTAGGCGTTAAAGATATAAACATTCAGGATCGTAAA |
| | primer | |
| SEQ ID | downstream | AAGCGCTATGGCATGCGCCATAGCATAATGACTGAACGTCCGATAAAAATAT |
| NO: 21 | bridge | ATAATA |
| | primer | |
| SEQ ID | downstream | AAGCGCTATGGCATGCGCCATAGCATAACGTTAAAGATATAAACATTCAGGA |
| NO: 22 | bridge | TCGTAAA |
| | primer | |
| SEQ ID | downstream | AAGCGCTATGGCATGCGCCATAGCATAATATATAATAGTTTAGGCGTTAAAG |
| NO: 23 | bridge | ATATAAACATTCAGGATCGTAAA |
| | primer | |
| SEQ ID | upstream | TTATGCTATGGCGCATGCCATAGCGCTTCTCTATACACTTGTTCAATT |
| NO: 24 | bridge | AACACAACCCGCATCATTTGATGTGGGAATGTCATTTTGC |
| | primer | |
| SEQ ID | upstream | TTATGCTATGGCGCATGCCATAGCGCTTCTCTATACACTTGTTCAATT |
| NO: 25 | bridge | AACACAACC |
| | primer | |
| SEQ ID | upstream | TTATGCTATGGCGCATGCCATAGCGCTTCGCATCATTTGATGTGGGAA |
| NO: 26 | bridge | TGTCATTTTGC |
| | primer | |
| SEQ ID | upstream | TTATGCTATGGCGCATGCCATAGCGCTTATTAACACAACCCGCATCAT |
| NO: 27 | bridge | TTGATGTGGGAATGTCATTTTGC |
| | primer | |
| SEQ ID | downstream | CGTTGCGATCAATGTTACCGTA |
| NO: 28 | primer | |
| SEQ ID | downstream | GTTGCGATCAATGTTACCGTAGTTT |
| NO: 29 | primer | |
| SEQ ID | downstream | AATTGAACGTTGCGATCAATGTTA |
| NO: 30 | primer | |

### Example 3

### Detection of the Pathogen Sequence by Generating and Detecting an Amplicon Combining Targets that are Located on Separate Genomes

In this example, the method of the present invention was successfully applied to generate an amplicon combining the sequences of two bacterial species: methicillin-sensitive Staphylococcus aureus (MSSA) and methicillin-resistant Staphylococcus epidermidis (MRSE). For this example, the primers were selected from Table 2, and the reaction conditions were identical to those in the second example. Results are shown in FIG. 7.

While the invention has been described in detail with reference to specific examples, it will be apparent to one skilled in the art that various modifications can be made within the scope of this invention. Thus the scope of the invention should not be limited by the examples described herein, but by the claims presented below.

We claim:

1. A method of amplifying a target nucleic acid in a sample, comprising:
a) contacting the target nucleic acid in the sample with a first pair of oligonucleotide primers flanking a first portion of the target sequence;
b) contacting the target nucleic acid in the sample with a second pair of oligonucleotide primers flanking a second portion of the target sequence, different from the first portion; wherein
one oligonucleotide primer in each pair is a bridge oligonucleotide containing a bridge sequence on the 5′-terminus; and
wherein the bridge sequence of the bridge oligonucleotide of the first pair is complementary to the bridge sequence of the bridge oligonucleotide of the second pair;
c) incubating the sample under the conditions permitting annealing of the oligonucleotide primers to the complementary sequences; and
d) incubating the sample under the conditions permitting extension of the oligonucleotide primers by the nucleic acid polymerase.

2. The method of claim 1, wherein steps c) and d) are repeated multiple times.

3. The method of claim 1, wherein the conditions in steps c) and d) are the same.

4. The method of claim 2 further comprising detecting the products of extension in step d).

5. The method of claim 1, wherein the target nucleic acid is derived from a pathogenic microorganism.

6. The method of claim 4, wherein the microorganism is selected from a group consisting of Staphylococcus and Mycobacterium.

7. The method of claim 1, wherein the target nucleic acid is derived from a mammal.

8. The method of claim 1, wherein the target nucleic acid is one or more of the genes EGFR, TP53, PDGF, PI3KCA, ERBB3, ERBB4, AKT1, KRAS, NF1, APC and STK11/LKB1.

9. A set of oligonucleotides for amplifying a target nucleic acid, comprising:
a) a first pair of oligonucleotides flanking a first portion of the target sequence,
b) a second pair of oligonucleotides flanking a second portion of the target sequence, different from the first portion; wherein
one oligonucleotide in each pair contains a bridge sequence on the 5′-terminus; and
wherein the bridge sequence of the oligonucleotide of the first pair is complementary to the bridge sequence of the oligonucleotide of the second pair.

10. The set of oligonucleotides of claim 6, further comprising one or more probe oligonucleotides.

11. The set of oligonucleotides of claim 6, wherein the one or more probe oligonucleotides are labeled.

12. A kit for amplifying a target nucleic acid comprising:
an amount of each of the oligonucleotides of a first pair, flanking a first portion of the target sequence;
an amount of each of the oligonucleotides of a second pair, flanking a second portion of the target sequence, different from the first portion;
wherein one oligonucleotide in each pair contains a bridge sequence on the 5′-terminus; and wherein the bridge sequence of the oligonucleotide of the first pair is complementary to the bridge sequence of the oligonucleotide of the second pair; and
a nucleic acid polymerase.

13. The kit of claim 9, further comprising one or more ucleoside triphosphates.

14. The kit of claim 9, further comprising one or more of the following: a pyrophosphatase, a uracil DNA glycosylase, a control nucleic acid and a set of instructions.

15. A reaction mixture for amplifying a target nucleic acid comprising:
a first pair of oligonucleotides flanking a first portion of the target sequence;
a second pair of oligonucleotides flanking a second portion of the target sequence, different from the first portion; a nucleic acid polymerase; and nucleoside triphosphates; and
wherein one oligonucleotide in each pair contains a bridge sequence on the 5′-terminus; and wherein the bridge sequence of the oligonucleotide of the first pair is complementary to the bridge sequence of the oligonucleotide of the second pair.

16. The reaction mixture of claim 12, further comprising one or more probe oligonucleotides.

17. The reaction mixture of claim 12, further comprising one or more of the following:
a pyrophosphatase and a uracil DNA glycosylase.

18. A method of generating a control molecule for an amplification reaction, the method comprising:
a) contacting a first target nucleic acid in the sample with a first pair of oligonucleotide primers flanking a portion of the first target sequence;
b) contacting a second target nucleic acid in the sample with a second pair of oligonucleotide primers flanking a portion of the second target sequence; wherein
one oligonucleotide primer in each pair is a bridge oligonucleotide containing a bridge sequence on the 5′-terminus; and
wherein the bridge sequence of the bridge oligonucleotide of the first pair is complementary to the bridge sequence of the bridge oligonucleotide of the second pair;
c) incubating the sample under the conditions permitting annealing of the oligonucleotide primers to the complementary sequences; and
d) incubating the sample under the conditions permitting extension of the oligonucleotide primers by the nucleic acid polymerase.

19. The method of claim 18, wherein steps c) and d) are repeated multiple times.

20. The method of claim 18, wherein the conditions in steps c) and d) are the same.

21. The method of claim 18 further comprising detecting the products of extension in step d).


SEQUENCE LISTING (30 sequences)

SEQ ID NO: 1 - DNA, 24 bp, Artificial Sequence
  Description of Artificial Sequence Synthetic primer
taacacatgcaagtcgaacggaaa

SEQ ID NO: 2 - DNA, 21 bp, Artificial Sequence
  Description of Artificial Sequence Synthetic primer
cgtgcttaacacatgcaagtc

SEQ ID NO: 3 - DNA, 23 bp, Artificial Sequence
  Description of Artificial Sequence Synthetic primer
tgcttaacacatgcaagtcgaac

SEQ ID NO: 4 - DNA, 22 bp, Artificial Sequence
  Description of Artificial Sequence Synthetic primer
atgcaagtcgaacggaaaggtc

SEQ ID NO: 5 - DNA, 52 bp, Artificial Sequence
  Description of Artificial Sequence Synthetic primer
aagcgctatggcatgcgccatagcataatcggattgacggtaggtggagaag

SEQ ID NO: 6 - DNA, 76 bp, Artificial Sequence
  Description of Artificial Sequence Synthetic primer
aagcgctatggcatgcgccatagcataatggtgcacattgcccaggaaattttcggattgacggtaggtggagaag

SEQ ID NO: 7 - DNA, 52 bp, Artificial Sequence
  Description of Artificial Sequence Synthetic primer
ttatgctatggcgcatgccatagcgcttttcgccactcgagtacctccgaag

SEQ ID NO: 8 - DNA, 76 bp, Artificial Sequence
  Description of Artificial Sequence Synthetic primer
ttatgctatggcgcatgccatagcgctttggtgcacattgcccaggaaatttttcgccactcgagtacctccgaag

SEQ ID NO: 9 - DNA, 27 bp, Artificial Sequence
  Description of Artificial Sequence Synthetic primer
cagttaagctgtgggatttcacgaaca

SEQ ID NO: 10 - DNA, 26 bp, Artificial Sequence
  Description of Artificial Sequence Synthetic primer
ttaagccgtgagatttcacgaacaac

SEQ ID NO: 11 - DNA, 25 bp, Artificial Sequence
  Description of Artificial Sequence Synthetic primer
tcacagttaagccgtgagatttcac

SEQ ID NO: 12 - DNA, 23 bp, Artificial Sequence
  Description of Artificial Sequence Synthetic primer
acgctcacagttaagccgtgaga

SEQ ID NO: 13 - DNA, 21 bp, Artificial Sequence
  Description of Artificial Sequence Synthetic probe
caactacgtgccagcagccgc

SEQ ID NO: 14 - DNA, 21 bp, Artificial Sequence
  Description of Artificial Sequence Synthetic probe
cgtgccagcagccgcggtaat

SEQ ID NO: 15 - DNA, 22 bp, Artificial Sequence
  Description of Artificial Sequence Synthetic probe
agcagccgcggtaatacgtagg

SEQ ID NO: 16 - DNA, 19 bp, Artificial Sequence
  Description of Artificial Sequence Synthetic primer
gatcaaacggcctgcacaa

SEQ ID NO: 17 - DNA, 22 bp, Artificial Sequence
  Description of Artificial Sequence Synthetic primer
cctgcacaaggacgtcttacaa

SEQ ID NO: 18 - DNA, 18 bp, Artificial Sequence
  Description of Artificial Sequence Synthetic primer
ggcctgcacaaggacgtc

SEQ ID NO: 19 - DNA, 21 bp, Artificial Sequence
  Description of Artificial Sequence Synthetic primer
cacaaggacgtcttacaacgc

SEQ ID NO: 20 - DNA, 96 bp, Artificial Sequence
  Description of Artificial Sequence Synthetic primer
aagcgctatggcatgcgccatagcataatgactgaacgtccgataaaaatatataatagtttaggcgttaaagatataaacattcaggatcgtaaa

SEQ ID NO: 21 - DNA, 58 bp, Artificial Sequence
  Description of Artificial Sequence Synthetic primer
aagcgctatggcatgcgccatagcataatgactgaacgtccgataaaaatatataata

SEQ ID NO: 22 - DNA, 59 bp, Artificial Sequence
  Description of Artificial Sequence Synthetic primer
aagcgctatggcatgcgccatagcataacgttaaagatataaacattcaggatcgtaaa

SEQ ID NO: 23 - DNA, 75 bp, Artificial Sequence
  Description of Artificial Sequence Synthetic primer
aagcgctatggcatgcgccatagcataatatataatagtttaggcgttaaagatataaacattcaggatcgtaaa

SEQ ID NO: 24 - DNA, 88 bp, Artificial Sequence
  Description of Artificial Sequence Synthetic primer
ttatgctatggcgcatgccatagcgcttctctatacacttgttcaattaacacaacccgcatcatttgatgtgggaatgtcattttgc

SEQ ID NO: 25 - DNA, 57 bp, Artificial Sequence
  Description of Artificial Sequence Synthetic primer
ttatgctatggcgcatgccatagcgcttctctatacacttgttcaattaacacaacc

SEQ ID NO: 26 - DNA, 59 bp, Artificial Sequence
  Description of Artificial Sequence Synthetic primer
ttatgctatggcgcatgccatagcgcttcgcatcatttgatgtgggaatgtcattttgc

SEQ ID NO: 27 - DNA, 71 bp, Artificial Sequence
  Description of Artificial Sequence Synthetic primer
ttatgctatggcgcatgccatagcgcttattaacacaacccgcatcatttgatgtgggaatgtcattttgc

SEQ ID NO: 28 - DNA, 22 bp, Artificial Sequence
  Description of Artificial Sequence Synthetic primer
cgttgcgatcaatgttaccgta

SEQ ID NO: 29 - DNA, 25 bp, Artificial Sequence
  Description of Artificial Sequence Synthetic primer
gttgcgatcaatgttaccgtagttt

SEQ ID NO: 30 - DNA, 24 bp, Artificial Sequence
  Description of Artificial Sequence Synthetic primer
aattgaacgttgcgatcaatgtta